(19) United States
(12) Patent Application Publication
(10) Pub. No.: US 2020/0093641 A1
(43) Pub. Date: Mar. 26, 2020

(54) SURGICAL INSTRUMENT, SURGICAL DEVICE, AND ELECTRONIC CONTROL DEVICE

(71) Applicant: Reinhardt Thyzel, Eckental (DE)

(72) Inventor: Reinhardt Thyzel, Eckental (DE)

(21) Appl. No.: 16/530,871

(22) Filed: Aug. 2, 2019

(30) Foreign Application Priority Data
Sep. 21, 2018 (DE) 20 2018 105 448

(51) Int. Cl. A61F 9/008

(52) U.S. Cl. CPC A61F 9/00825; A61F 2009/00874; A61B 2218/007

(57) ABSTRACT

A surgical instrument for vitrectomy, in particular vitrectomy device or vitrector, comprising a hollow needle, in particular cannula, with a cylindrical wall which encloses a cavity of the hollow needle, wherein the hollow needle is closed on the end face at a distal end located in the longitudinal direction (L) of the hollow needle, and the hollow needle comprises a vitrectomy opening, whose opening normal is oriented radially or transversely to the longitudinal direction (L), has a predetermined minimum distance from the distal end, and the opening cross-sectional area in the longitudinal direction (L), or transversely to the longitudinal direction, or in the circumferential direction (U) comprises an elongated, at least partially curved shape, in particular a shape formed in the manner of an ellipse.

### CROSS-REFERENCE TO RELATED APPLICATIONS

This application claims the benefit of priority to German Patent Application No. DE 20 2018 105 448, filed on Sep. 21, 2018, entitled “Surgical Instrument, Surgical Device, and Electronic Control Device,” the entire content of which is incorporated herein by reference.

### BACKGROUND OF THE INVENTION

### 1. Technical Field

In particular, the invention relates to a surgical instrument for vitrectomy, in particular a vitrectomy device or a vitrector, a surgical device with such a surgical instrument, as well as an associated electronic control device.

### 2. Background and Relevant Art

During vitrectomy, parts of the vitreous body of the eye are removed, which can be necessary, for example, for blood caused clouding or retinal detachment. Due to the relatively firm consistency of the vitreous body material, it is not possible to suck it away in a simple manner in vivo.

From the prior art, for example from WO 2015/135544 A1, vitrectors are known which comprise mechanical moving cutting elements for the separation of vitreous material in vivo. Such vitrectors can be disadvantageous in that mechanical kinetic energy of the moving cutting elements, e.g. in the form of vibrations, is transmitted to the eye, in particular the retina, and leads amongst other things to damage or impairment. Apart from that, it would be desirable to increase the efficiency of removing vitreous material in vivo over known mechanical vitrectors.

### BRIEF SUMMARY OF THE INVENTION

Accordingly, it can be regarded as an object of the invention to provide a surgical instrument for vitrectomy, in particular a vitrector, a surgical device, and a corresponding electronic control device which enables an efficient and conservative (or gentle) removal of parts of the vitreous body of the eye, in particular in vivo.

This object is achieved by the subject matter of the independent claims. Advantageous embodiments will be apparent from the dependent claims. Embodiments also result from the following description.

According to embodiments, a surgical instrument for vitrectomy, in particular a vitrectomy device or a vitrector, in particular a vitrectomy needle, is provided.

The surgical instrument comprises a hollow needle, in particular cannula, with a cylindrical wall which encloses a cavity, in particular inner cavity, of the hollow needle. The hollow needle is closed on the end face at a distal end located in the longitudinal direction of the hollow needle, e.g. in a dome or capsule shaped manner.

The hollow needle comprises a vitrectomy opening, i.e. an opening specially designed for the purpose of vitrectomy.

The opening normal of the vitrectomy opening, in particular the normal of the opening cross-sectional area of the vitrectomy opening viewed in plan view, is oriented radially or transversely to the longitudinal direction of the hollow needle.

Furthermore, the vitrectomy opening has a predetermined minimum distance from the distal end, in particular from the outer distal end, of the hollow needle, that is closed on its front face.

Furthermore, the opening cross-sectional area of the vitrectomy opening, in particular the opening cross-sectional area of the vitrectomy opening when viewed in plan view, has an elongated, at least partially curved shape, in particular an elongated shape formed in the manner of some kind of (true) ellipse, when viewed longitudinally or transversely to the longitudinal direction, for example perpendicular to the longitudinal direction or in the circumferential direction with respect to the longitudinal direction of the hollow needle. For example, the peripheral edge defined by the vitrectomy opening, in particular when viewed in plan view, may have an elongated, at least partially curved shape, in particular an elongate shape formed in the manner of some kind of (true) ellipse.

With such a vitrectomy opening a removal, in particular comprising breaking up or cutting and sucking away, of parts of the vitreous body of the eye, is possible in an efficient manner, in particular in vivo, e.g. in connection with a laser beam-induced breaking up of vitreous material, that has entered the interior of the hollow needle via the vitrectomy opening, e.g. upon exposure of the interior of the hollow needle with a negative pressure.

In embodiments, the hollow needle may be formed, for example, single-walled. Furthermore, the vitrectomy opening may be formed in a wall or the, in particular single-walled, shell wall (or: cylindrical wall) of the hollow needle.

The vitrectomy opening may be communicating, in particular fluidly communicating, with a chamber immediately adjoining the vitrectomy opening and the distal end. In particular with such a vitrectomy opening, vitreous material can be efficiently, yet conservatively (or gently) removed in vivo, in particular sucked away.

In embodiments, a light guide, in particular a laser light guide device or a laser optical system, may be arranged in the interior of the hollow needle for introducing laser light, in particular laser pulses, preferably Nd:YAG laser pulses, into the chamber. The light guide may in particular comprise an optical fiber and/or a light-conducting optic or photo-optical fiber optic.

The light exit side or surface of the light guide or the light exit side or surface of a light optic of the light guide can, according to embodiments, be facing the chamber, in particular such that glass body material that entered the chamber via the opening may be exposed to laser pulses for the purpose of breaking it up. For example, the light exit side may be arranged such that laser light, in particular laser pulses, may be delivered with a preferred direction directed in the longitudinal direction of the hollow needle, and such that respectively vitreous material that is present in the chamber in the longitudinal direction of the light exit side, e.g. in direction towards the distal end, may be separated, broken up or cut.

In embodiments, the hollow needle may further comprise in its interior a suction channel (or: aspiration channel), preferably extending at least partially parallel to the light guide. The suction channel may in particular be designed such that glass body material may be sucked out of the chamber, in particular out of the inner cavity of the hollow needle, after being exposed to laser light, that is to say after separation has taken place. In embodiments, the suction channel may be formed directly communicating, i.e. fluidly communicating, with the chamber.

In embodiments, the distance between the opening edge of the vitrectomy opening facing the outer end face of the distal end, that is closed on its front face, on the one hand, and the outer end face of the distal end, that is closed on its front face, of the hollow needle, on the other hand, may be in the range from 0.4 mm to 1.8 mm, preferably in the range from 0.5 mm to 1.6 mm, more preferably in the range of 0.55 mm to 1.55 mm.

In embodiments, the distance between the outer end face of the distal end, that is closed on its front face, of the hollow needle on the one hand and the central point (or: central axis) of the opening cross-sectional area of the vitrectomy opening measured in the longitudinal direction of the hollow needle on the other hand, may be in the range of 0.4 mm to 1.8 mm, preferably in the range from 0.5 mm to 1.6 mm, more preferably in the range of 0.55 mm to 1.55 mm.

In embodiments, the wall thickness of the distal end, that is closed on its front face, of the hollow needle measured parallel to the longitudinal direction may be in the range of 0.25 mm to 0.85 mm, preferably in the range of 0.3 mm to 0.8 mm.

The aforementioned dimensions allow in particular for an efficient and conservative (or gentle) sucking away of vitreous material.

In embodiments, the opening cross-sectional area, i.e. the cross-sectional area of the vitrectomy opening or a corresponding opening edge, comprises, viewed in plan view (in particular: in plan view in the radial direction to the longitudinal axis), a rounded shape, preferably a substantially circular segment-like or circular arc-like, in particular semicircular arc-like shape, at least on one longitudinal end of the vitrectomy opening, preferably at both longitudinal ends of the vitrectomy opening.

The term “longitudinal end” is intended in particular to designate an end located in the longitudinal extent of the vitrectomy opening, for example defined by the edge of the vitrectomy opening in the lateral surface of the hollow needle.

In embodiments, opening edges may extend between the longitudinal ends, i.e. between the two ends lying in the longitudinal extent of the vitrectomy opening, at least partially rectilinear and/or parallel, preferably they may be formed straight-lined parallel. In particular, the at least partially rectilinear opening edges, or corresponding opening edge portions, may extend parallel to the longitudinal direction of the hollow needle or transversely, in particular perpendicular, to the hollow needle.

The mentioned opening geometries show advantages, in particular in terms of the entry of vitreous material into the chamber, in particular in combination with laser-induced breaking up, and allow efficient removal of vitreous material in vivo, for example.

In embodiments, the opening cross-sectional area, particularly in plan view, may be ellipse-like, e.g. like a real ellipse, or be designed in the manner of a slot (hole) (or elongated or slotted hole).

In embodiments, the hollow needle (especially at least partially, or substantially) may be made of titanium, in particular titanium grade 4, i.e. the needle may be made (at least partially) of titanium. Furthermore, the hollow needle, in particular the wall of the hollow needle, may be formed as an integral unit, i.e. formed from a single material or made out of it.

In embodiments, the hollow needle may have a length of at least or at most 25 mm, in particular of at least or at most 30 mm.

Furthermore, in embodiments, the hollow needle may comprise, in particular in the region of the vitrectomy opening, an outer diameter in the range from 0.35 mm to 0.75 mm, preferably 0.4 mm to 0.7 mm, in particular 0.65 mm.

Furthermore, in embodiments, the shell wall of the hollow needle may comprise, in particular in the region of the vitrectomy opening, a thickness in the range of 0.1 mm to 0.3 mm, preferably from 0.15 mm to 0.25 mm, more preferably of 0.2 mm.

The aforementioned hollow needle dimensions are particularly suitable for an efficient and conservative (or gentle) performance of a vitrectomy.

In embodiments, the vitrectomy opening, in particular its opening cross-sectional area, may comprise a length in the range of 0.5 mm to 0.8 mm, in particular 0.55 mm to 0.75 mm, in particular 0.65 or 0.6 mm, and a width in the range of 0.25 mm to 0.35 mm, in particular 0.3 mm. Corresponding vitrectomy openings, particularly in conjunction with laser exposure of the vitreous material in the chamber, for example with a preferential laser propagation direction parallel to the longitudinal axis, allow a particularly efficient removal of vitreous material, in particular with regard to removal rates or extraction (sucking away) rates.

In embodiments, the outer surface of the end face of the distal end, that is closed on its front face, of the hollow needle may be curved, e.g. dome-like or capsule-like, at least in sections. Furthermore, in embodiments, the outer surface may have a radius of curvature in the range of 0.15 mm to 0.35 mm, preferably 0.2 mm to 0.3 mm, at least in sections. Such needle geometries are particularly suitable for carefully or gently carrying out a vitrectomy.

In embodiments, the opening edge of the hollow needle, in particular viewed in plan view, may be at least partially curved with a radius of curvature in the range of 0.1 mm to 0.2 mm, preferably 0.15 mm. Such edge geometries have proved to be particularly suitable in that in an in vivo vitrectomy, vitreous material may be sucked and removed comparatively efficiently into the chamber adjoining the vitrectomy opening.

In embodiments, a surgical device, in particular a vitrectomy device, may be provided which comprises a surgical instrument according to an embodiment described herein, in particular according to an embodiment according to the attached claims.

The surgical device may comprise a laser light source, in particular a Nd:YAG laser light source, for example a Q-switched Nd:YAG laser source, for coupling laser light, in particular laser pulses, into the cavity, in particular into the chamber of the hollow needle.

For coupling the laser light into the cavity, e.g. into the chamber fluidically communicating (directly) with the vitrectomy opening, the device may comprise a light guide, in particular a laser light guide. The light guide may be arranged, e.g. at least in partially, inside the hollow needle.

The laser light source, in particular a control unit for controlling the laser light source, may be configured in embodiments to generate laser light with a pulse frequency in the range of 40 Hz to 60 Hz. The laser light source may be optically coupled to the light guide or a suitable light optics or be capable of being coupled, such that laser light, in particular laser pulses, generated by the laser light source may be passed via the light guide or the light optics in the chamber.

In embodiments, the device may further comprise a suction unit (in particular: aspiration unit) which is coupled or may be coupled to the inner cavity of the hollow needle. In embodiments, the suction unit may be set up and operated such that the cavity of the hollow needle may be subjected to negative pressure. In particular, the suction unit may be set up such that the cavity, in particular the chamber fluidly coupled to the vitrectomy opening, may be exposed to a negative pressure in the range of 0.2 bar (20 kPa) to 0.4 bar (40 kPa), preferably 0.25 bar (25 kPa) to 0.35 bar (35 kPa), more preferably from 0.26 bar (26 kPa) to 0.33 bar (33 kPa).

In embodiments, an electronic control device may be provided with a control interface for the control coupling with a surgical instrument according to an embodiment described herein, in particular according to an embodiment of the claims, and/or with a surgical device according to an embodiment described herein, in particular an embodiment of the claims.

In embodiments, the control device may comprise control means, in particular in the form of hardware and/or in the form of instructions stored on or in memory for execution by an electronic computer or data processing unit, the execution of which in the operation of the control device produce at least one of the following process steps in the surgical instrument or in the surgical device: generating laser pulses, in particular with a laser pulse frequency in the range of 40 Hz to 60 Hz, wherein the laser pulses are coupled into the inner cavity of the hollow needle, in particular the chamber, via a light guide or a light optics, and emerge at a light exit side of the light guide or the light optics, e.g. with a preferred direction parallel to the longitudinal direction of the hollow needle, such that glass body material of the eye located in a chamber or the chamber formed inside the hollow needle, is broken up, in particular cut, by exposure to the laser impulse. In this case, the chamber is designed to communicate fluidically with a vitrectomy opening formed according to a embodiment described herein, in particular a vitrectomy opening constructed in accordance with a claimed embodiment; applying (or exposing) to the inner cavity of the hollow needle, in particular the chamber, negative pressure, in particular in the range of 0.2 bar to 0.4 bar, preferably from 0.25 bar to 0.35 bar, more preferably from 0.26 bar to 0.33 bar, such that the vitreous material broken up by the laser pulses, is in particular sucked away by the effect of the negative pressure, and at the same time further vitreous material enters the chamber when the vitrectomy opening corresponding according to an embodiment described herein, in particular a claimed embodiment, is positioned, especially in vivo, in the vitreous body of an eye, e.g. a human or animal eye.

### BRIEF DESCRIPTION OF THE DRAWINGS

Hereinafter, embodiments of the invention will be described in more detail with reference to the accompanying drawings.

FIG. 1 shows a vitrector in a first embodiment in plan view;

FIG. 2 shows the vitrector of FIG. 1 in cross section;

FIG. 3 shows a vitrector in a second embodiment in plan view;

FIG. 4 shows the vitrector of FIG. 3 in cross-section;

FIG. 5 shows a top view of a vitrector in a third embodiment;

FIG. 6 is a detail of the vitrector of FIG. 5;

FIG. 7 shows a cross section of the vitrector according to FIG. 5; and

FIG. 8 is a partial perspective view of the vitrector of FIG. 6.

### DETAILED DESCRIPTION FO THE PREFERRED EMBODIMENTS

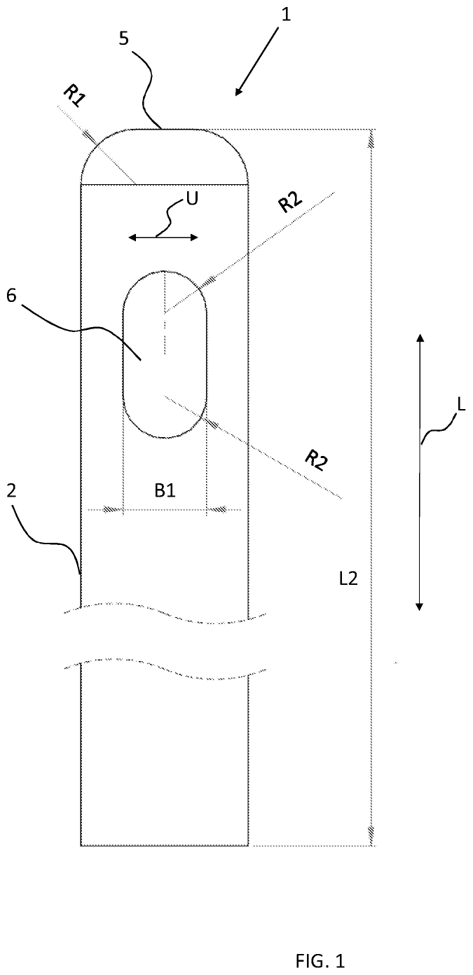

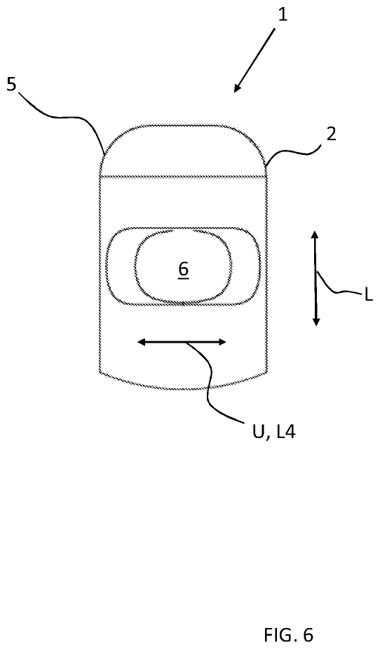

Corresponding parts and sizes are provided with the same reference numerals in FIGS. 1 to 7.

FIGS. 1 to 8 show exemplary embodiments of a surgical instrument, in particular a vitrector 1 or a vitrectomy needle for carrying out a vitrectomy, in particular on the eye in vivo.

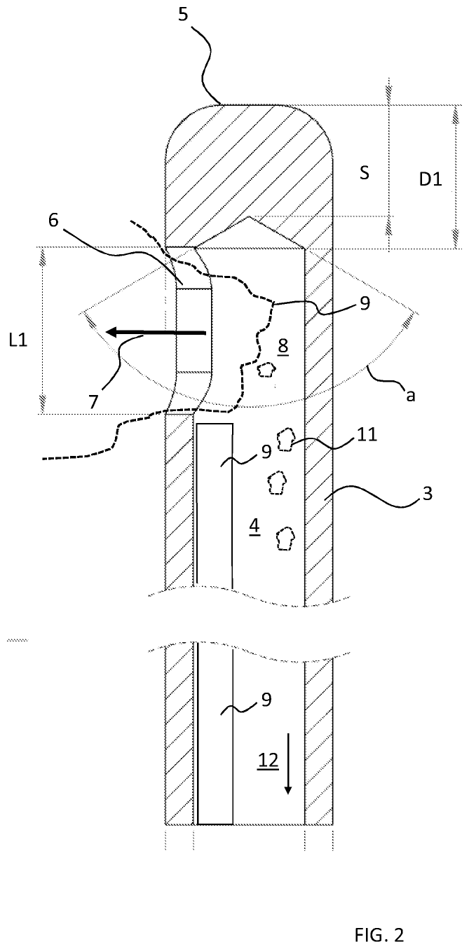

The vitrector 1 according to FIG. 1 comprises a hollow needle 2, e.g. in the manner of a cannula. The hollow needle 2 comprises a cylindrical wall 3, which is clear in particular in conjunction with FIG. 2, which encloses a cavity 4 of the hollow needle 2.

The hollow needle 2 is closed on the front face at a distal end 5 located in the longitudinal direction L of the hollow needle 2.

The hollow needle 2 further comprises a vitrectomy opening 6, wherein the opening normal 7 of the vitrectomy opening 6 is oriented radially to the longitudinal direction L. In the given embodiment the vitrectomy opening 6 is distanced 0.42 mm from the distal end 5. More specifically, in the present embodiment, the distance D1 between the distal end 5 on the one hand and the opening edge of the vitrectomy opening facing the distal end 5 on the other hand is 0.42 mm.

The opening cross-sectional area of the vitrectomy opening 6, in particular viewed in plan view of FIG. 1, comprises in the present embodiment in the longitudinal direction L, an elongated, partially curved shape, in particular in the manner of a slot. In particular, both sides of the opening edge are semicircular, and connected by rectilinear edge portions.

Dimensions and geometrical details for the vitrector 1 or the hollow needle 2 as well as for the vitrectomy opening 6 are shown in particular in FIGS. 1 and 2.

Thus, in the given embodiment, the vitrectomy opening 6 measured in the circumferential direction U has a width B1 of 0.3 mm, and measured in the longitudinal direction L has a length L1 of 0.6 mm. The total length L2 of the hollow needle is about 25 mm.

Curvature radii R2 for the semicircular opening edges may be, for example, 0.15 mm. Roundings in the region of the distal end 5 can, for example, have radii of curvature R2 of 0.2 mm. For example, the outer shape of the distal end 5 may be formed like a dome. Other forms are also possible.

As may be seen from the cross-sectional illustration of FIG. 2, the thickness S of the wall 3 of the hollow needle 2 at the distal end 5, measured in the direction of the longitudinal axis of the hollow needle 2, may be in the range of 0.3 mm.

The hollow needle 2 as such can, for example, in particular be made as one piece out of solid material Ti-grade 4, wherein the cavity 4 may be made by drilling, so that an outer diameter of 0.6 mm results in an inner diameter of 0.4 mm.

By using a drill with a conical drill tip, an opening angle a, which is 120 degrees in the present example, is generated at the end of the cavity 4 located at the distal end. However, other opening angles are possible. In particular, it is also possible, for example when using a drill with non-conical drill bit or in other methods for producing the cavity, that the distal end inner wall of the cavity is straight, in particular perpendicular to the longitudinal direction L, or convexly or concavely curved.

As is apparent from FIG. 2, the hollow needle 2 is single-walled and the vitrectomy opening 6 is formed in the shell wall, i.e. the cylindrical wall 3, the hollow needle 2.

The vitrectomy opening 6 is formed fluidly communicating immediately adjacent to a chamber 8 located inside the hollow needle 2. In other words, in the area of the vitrectomy opening 6, the inner cavity 4 of the hollow needle 2 forms a chamber 8, which is adapted to receive vitreous material 9 (indicated in FIG. 2 by dotted line) entering through the vitrectomy opening, e.g. during in vivo vitrectomy of the eye.

By means of a light optic, in particular a photo-optical fiber 10, laser light, in particular in the form of laser pulses, for example with a frequency of 40 Hz to 60 Hz, may be radiated into the chamber 8 by means of a laser device (not shown), for example with a preferred direction extending parallel to the longitudinal direction. By the exposure to the laser light, the glass body material 9 located in the chamber 8 may be broken up, in particular “cut”. In particular, glass body material pieces 11 may be separated.

Separated vitreous material pieces 11 may be removed through a suction channel 12 formed in a cavity 4 of the hollow needle 2, e.g. in the direction of the arrow associated with reference numeral 12, i.e. away from the distal end.

For sucking away the vitreous material pieces 11, it is possible to apply a negative pressure of e.g. 200 to 250 mmHg (about 26.7 kPa to 33.3 kPa) to the hollow needle 2, i.e. to the cavity 4. The negative pressure causes, for example, that vitreous material 9, e.g. during in vitro vitrectomy, is sucked through the vitrectomy opening 6 into the chamber 8, so that it may be exposed to laser radiation, in particular laser pulses. The laser radiation or the laser pulses may be generated as mentioned, for example with a Nd:YAG laser.

In the embodiment of FIG. 2, the light exit side or light exit surface of the light guide 9 or the light optics is facing in the direction of the distal end 5 of the chamber 8.

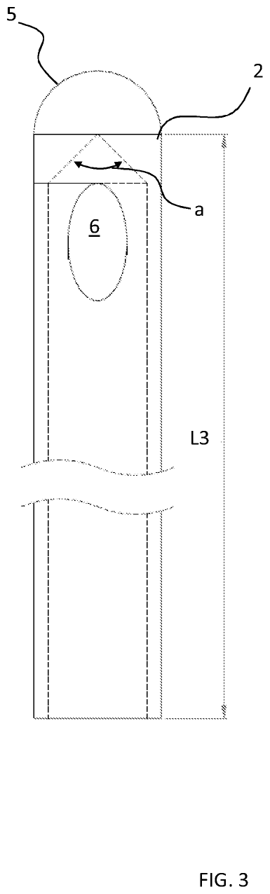

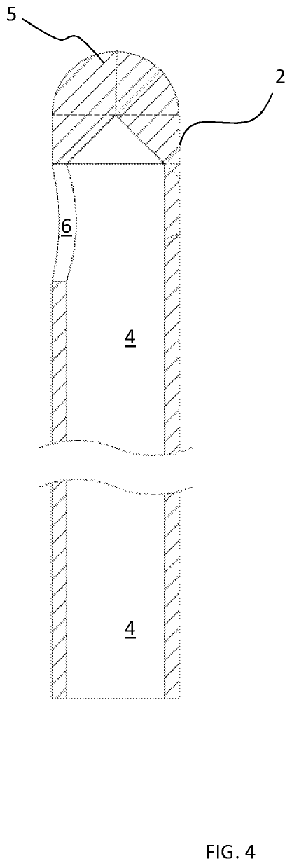

FIGS. 3 and 4 show a second exemplary embodiment of a hollow needle 2. In the following, only the essential differences between the hollow needle of FIG. 1 and FIG. 2 will be discussed. Unless otherwise stated in the following description, in connection with the second and third exemplary embodiment elements and components not discussed in detail, or elements and components not shown in the figures, may basically be constructed and arranged the same as in the first embodiment.

In particular, the vitrectomy opening 6 of FIG. 3 differs from that of FIG. 1 in the specific geometric shape, in particular the shape of the opening cross-sectional area. While the vitrectomy opening 6 of FIG. 1 is designed in the manner of a slot, the vitrectomy opening 6 of FIGS. 3 and 4, viewed in plan view, is designed as an ellipse or ellipse-like in the direction of the longitudinal axis L of the hollow needle 2. Such a shape has also proved to be particularly suitable for the efficient performance of a vitrectomy in addition to the form shown in FIGS. 1 and 2.

Differences further exist in the opening angle a of the longitudinal bore forming the cavity 4 in the region of the distal end 5. The opening angle a is by way of example approximately 90 degrees in the example of FIG. 3, but may also be different. For example, the front end surface of the cavity 4 or the chamber 8 at the distal end 5 may also be, for example, straight (i.e. transverse or perpendicular to the longitudinal axis), or convexly or concavely curved.

Another difference is in the outer shape of the distal end 5 of the hollow needle 2. In the embodiment of FIGS. 3 and 4, the shape of the outer distal end 5 is dome-shaped or cupola-shaped, in its cross-section in particular a semicircular or a segment of a circle. The length L3 of the cylindrical portion of the hollow needle 2, i.e. without a curved distal end, may e.g. be about 25 mm.

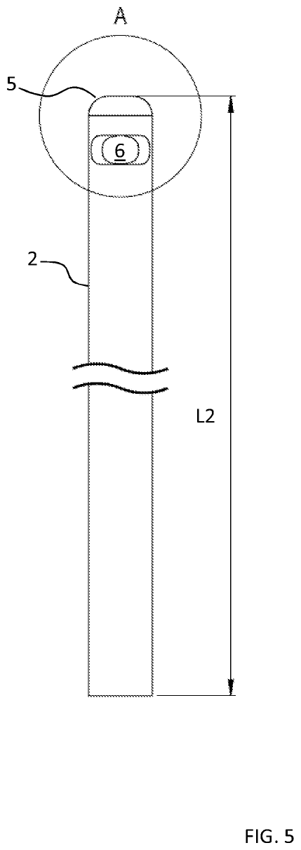

FIGS. 5 to 8 show views of a third exemplary embodiment of a vitrector 1 or a hollow needle 2. The difference between the vitrector 1 to the first and second embodiments lies in particular in the orientation or geometric extent of the vitrectomy opening 6.

In the third embodiment, the vitrectomy opening 6, as in the other embodiments, comprises an advantageous elongated shape (in particular viewed in plan view), but the expansion of the vitrectomy opening measured in the circumferential direction U is, in contrast to the first and second embodiments, greater than their extension in Longitudinal direction L.

The opening area of the vitrectomy opening 6 considered in plan view corresponds approximately to the shape of a slot oriented in the circumferential direction U, with opening edges, curved at least at the opposite ends, for example circular-like or alternatively also circularly curved.

The shape of the vitrectomy opening 6 shown in FIGS. 5 to 8 also permits a particularly efficient and conservative (or gentle) vitrectomy.

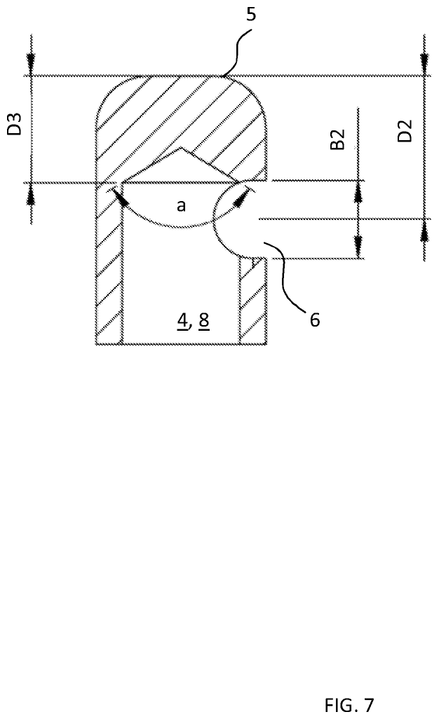

Some exemplary dimensions are shown in FIG. 7. For example, the inner diameter of the cylindrical portion of the hollow needle 2 may be 0.45 mm.

The width B2 of the vitrectomy opening 6, measured in the longitudinal direction L, can, for example be 0.3 mm, wherein the length L4 measured in the circumferential direction U may for example be about 0.6 mm.

A distance D2 between the outer distal end 5 of the hollow needle 2 and the central axis of the vitrectomy opening 6, in particular the central axis of the opening area of the vitrectomy opening 6, may for example be 0.55 mm, wherein a distance D3 between the outer distal end 5 of the hollow needle 2 and the opening edge facing the distal end 5 may for example be 0.41 mm.

An opening angle a of the longitudinal bore at the distal end 5 may be for example 118 degrees. However, the opening angle a may also be different, e.g. depending on the drill used. The end face of the cavity 4 or of the chamber 8 which is located at the distal end 5 may alternatively also be straight or planar, or curved, e.g. be curved convexly or concavely.

Although not all features of the first embodiment are shown in connection with the second and third embodiments, e.g. the photo-optical fiber 10, they are correspondingly present in the functional embodiment of the hollow needles 2.

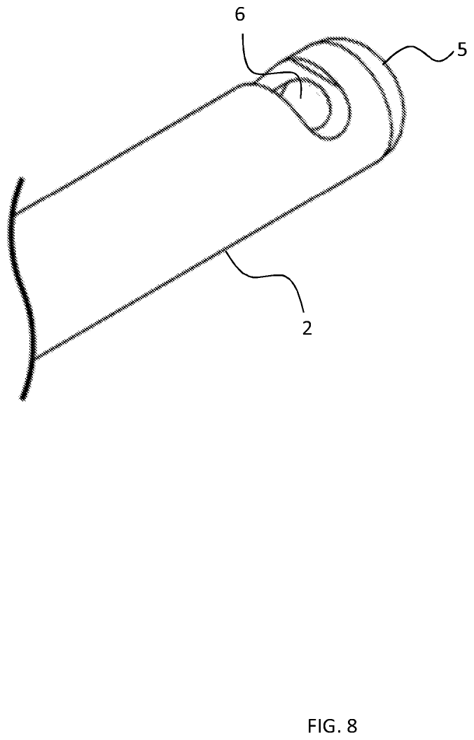

Finally, FIG. 8 also shows a perspective view of the hollow needle 2 of the third embodiment, from which the vitrectomy opening 6 with its longitudinal axis extending in the circumferential direction U can clearly be seen. In particular, with a vitrectomy opening having a circumferential axis extending in the circumferential direction U, i.e. the length of the vitrectomy opening 6 in the circumferential direction is greater than the width in the direction of the longitudinal axis L, in combination with laser pulses, which are substantially emitted in the longitudinal direction in the chamber 8, a comparatively efficient vitrectomy result, in particular with regard to the removal rates, can be achieved.

According to the three exemplary embodiments, a herein proposed vitrector 1 may in particular have an opening cross-sectional area which, viewed in plan view, has a rounded shape, preferably a substantially circular segment-like or circular arc-like, in particular semicircular, form, and/or it may be provided at both longitudinal ends that the opening edges extending between the longitudinal ends are at least partially rectilinear and/or parallel, preferably formed in a straight-line parallel. In particular, the opening cross-sectional area, viewed in plan view, may be formed in the manner of an ellipse, or in the manner of a slot, in particular in the manner of slot different from a (defined by straight opening edges) slot. The vitrectomy opening may, for example, be formed by drilling or milling.

Furthermore, it is possible that the opening edge of the hollow needle 2, in particular viewed in plan view, is curved at least in sections with a radius of curvature R2 in the range of 0.1 mm to 0.2 mm, preferably 0.15 mm.

The hollow needles 2 shown in the first to third embodiments may be part of a surgical device, which, as described above, further may comprise a laser light source, in particular a Nd:YAG laser light source, for coupling laser light, in particular laser pulses, into the cavity 4 or the chamber 8 of the hollow needle 2, in particular via the photo-optical fiber 10 arranged at least partially in the interior of the hollow needle 2. The laser light source can, for example, comprise a control unit for controlling the laser light source, which is adapted to generate laser light with a pulse frequency in the range of 40 Hz to 60 Hz and to feed it into the chamber 8.

Furthermore, such a device may comprise a suction unit (not shown) coupled to the cavity 4 and the chamber 8 of the hollow needle 2. The suction unit can, e.g. be set up and operated so that the cavity 4 in particular the chamber 8 of the hollow needle 2 may be acted upon by negative pressure, in particular by a negative pressure in the range of 200 mmHg to 250 mmHg.

Furthermore, an electronic control device may be provided with a control interface for the control technology coupling with a corresponding surgical instrument, wherein the control device may comprise control means whose execution during operation of the control device in the surgical instrument 1 or the surgical device may cause at least one of the method steps already described above.

Overall, it may be seen that the surgical instrument, the surgical device or the control device proposed herein solve the underlying problem.

### LIST OF REFERENCE NUMBERS

1 vitrector
2 hollow needle
3 cylindrical wall
4 cavity
5 distal end
6 vitrectomy opening
7 opening normal
8 chamber
9 vitreous material
10 photo-optical fiber
11 vitreous body pieces
12 suction channel
a opening angle
Bx widths
Dx distances
L lengthwise direction
Lx: lengths
U circumferential direction
Rx radii of curvature
S thickness

I claim:

1. A surgical instrument for vitrectomy, in particular vitrectomy device or vitrector, comprising:
a hollow needle, in particular a cannula, with a cylindrical wall which encloses a cavity of the hollow needle;
wherein the hollow needle is closed on the end face at a distal end located in the longitudinal direction (L) of the hollow needle; and
wherein the hollow needle comprises a vitrectomy opening;
wherein the vitrectomy opening has:
an opening normal that is oriented radially or transversely to the longitudinal direction (L);
a predetermined minimum distance from the distal end; and
a cross-sectional area (i) in the longitudinal direction (L), or (ii) transversely to the longitudinal direction, or (iii) in the circumferential direction (U), which comprises an elongated, at least partially curved shape, in particular a shape formed in the manner of an ellipse.

2. The surgical instrument according to claim 1, wherein:
the hollow needle is of single-walled construction; and/or
the vitrectomy opening is formed in the shell wall of the hollow needle and is formed communicating with a chamber immediately adjacent to the vitrectomy opening and to the distal end.

3. The surgical instrument according to claim 2, wherein:
a light conductor designed to guide laser light, in particular laser pulses, preferably Nd:YAG laser pulses, into the chamber is arranged inside the hollow needle;
the light exit side of the light guide or of a light optics of the light guide is facing the chamber; and
the hollow needle further comprises in the interior a, preferably at least partially parallel to the light guide extending, suction channel;
the suction channel in particular is adapted for sucking away glass body material from the chamber after exposure to laser pulses; and
the suction channel is formed directly communicating with the chamber.

4. The surgical instrument according to claim 1, wherein:
the distance (D1, D3) between the opening edge of the vitrectomy opening facing the distal end, that is closed on its front face, and the distal end, that is closed on its front face, of the hollow needle is in the range of 0.4 mm to 1.8 mm, preferably in the range of 0.5 mm to 1.6 mm, more preferably in the range of 0.55 mm to 1.55 mm; and/or
the distance (D2) between the outer, distal end, that is closed on its front face, of the hollow needle and the center of the opening cross-sectional area of the vitrectomy opening measured in the longitudinal direction (L) of the hollow needle is in the range from 0.4 mm to 1.8 mm, preferably in the range of 0.5 mm to 1.6 mm, more preferably in the range of 0.55 mm to 1.55 mm; and/or
the wall thickness (S) of the distal end, that is closed on its front face, of the hollow needle measured parallel to the longitudinal direction (L) is in the range of 0.25 mm to 0.85 mm, preferably in the range of 0.3 mm to 0.8 mm.

5. The surgical instrument according to claim 1, wherein:
the opening cross-sectional area viewed in plan view comprises at least one longitudinal end, preferably at both longitudinal ends, a rounded shape, preferably a substantially circular segment-like or circular arc-like, in particular semicircular arc-like shape; and/or
opening edges extending between the longitudinal ends are formed at least partially rectilinear and/or parallel, preferably straight-lined parallel.

6. The surgical instrument according to claim 5, wherein the opening cross-sectional area, in particular viewed in plan view, is designed in the manner of an ellipse, or in the manner of a slot.

7. The surgical instrument according to claim 1, wherein:
the hollow needle is made of titanium, in particular titanium grade 4, and/or
the hollow needle comprises a length (L2) of at least 25 mm, in particular 30 mm; and/or
the hollow needle comprises, in particular in the region of the vitrectomy opening, an outer diameter in the range of 0.35 mm to 0.75 mm, preferably 0.4 mm to 0.7 mm, in particular 0.65 mm; and/or
the shell wall of the hollow needle comprises, in particular in the region of the vitrectomy opening, a thickness in the range of 0.1 mm to 0.3 mm, preferably from 0.15 mm to 0.25 mm, more preferably from 0.2 mm.

8. The surgical instrument according to claim 1, wherein:
the vitrectomy opening, in particular its opening cross-sectional area, has a length (L1, L4) in the range from 0.5 mm to 0.8 mm, in particular 0.55 mm to 0.75 mm, in particular 0.65 mm or 0.6 mm, and/or a width (B1, B2) in the range of 0.25 mm to 0.35 mm, in particular 0.3 mm.

9. The surgical instrument according to claim 1, wherein:
the outer surface of the distal end, that is closed on its front face, of the hollow needle is curved at least in sections; and
the outer surface optionally comprises at least in sections a radius of curvature (R1) in the range of 0.15 mm to 0.35 mm, preferably 0.2 mm to 0.3 mm.

10. The surgical instrument according to claim 1, wherein the opening edge of the hollow needle, in particular viewed in plan view, is at least partially curved with a radius of curvature (R2) in the range of 0.1 mm to 0.2 mm, preferably 0.15 mm.

11. A surgical device, in particular a vitrectomy device, comprising:
the surgical instrument comprising a hollow needle, in particular a cannula, with a cylindrical wall which encloses a cavity of the hollow needle;
wherein the hollow needle is closed on the end face at a distal end located in the longitudinal direction (L) of the hollow needle; and
wherein the hollow needle comprises a vitrectomy opening;
wherein the vitrectomy opening has:
an opening normal that is oriented radially or transversely to the longitudinal direction (L);
a predetermined minimum distance from the distal end; and
a cross-sectional area (i) in the longitudinal direction (L), or (ii) transversely to the longitudinal direction, or (iii) in the circumferential direction (U), which comprises an elongated, at least partially curved shape, in particular a shape formed in the manner of an ellipse; and
a laser light source, in particular a Nd:YAG laser light source, for coupling laser light, in particular laser pulses, into the cavity, in particular into the chamber, of the hollow needle, in particular via a light guide arranged at least partially in the interior of the hollow needle;
wherein the laser light source, in particular a control unit for controlling the laser light source, is arranged to produce laser light with a pulse frequency in the range of 40 Hz to 60 Hz, and optionally further comprises a suction unit that is coupled or coupleable to the inner cavity of the hollow needle;
wherein the suction unit is preferably set up and operable in such a way, that the cavity, in particular the chamber, of the hollow needle may be acted upon by negative pressure, in particular by a negative pressure in the range of 0.2 bar to 0.4 bar, preferably from 0.25 bar to 0.35 bar, more preferably from 0.26 bar to 0.33 bar.

12. An electronic control device with a control interface for controlling coupling with a surgical instrument comprising:
a hollow needle, in particular a cannula, with a cylindrical wall which encloses a cavity of the hollow needle;
wherein the hollow needle is closed on the end face at a distal end located in the longitudinal direction (L) of the hollow needle; and
wherein the hollow needle comprises a vitrectomy opening;
wherein the vitrectomy opening has:
an opening normal that is oriented radially or transversely to the longitudinal direction (L);
a predetermined minimum distance from the distal end; and
a cross-sectional area (i) in the longitudinal direction (L), or (ii) transversely to the longitudinal direction, or (iii) in the circumferential direction (U), which comprises an elongated, at least partially curved shape, in particular a shape formed in the manner of an ellipse; and
control means whose execution during operation of the control device cause the following process steps in the surgical instrument or the surgical device:
generation of laser pulses, in particular with a laser pulse frequency in the range of 40 Hz to 60 Hz, the laser pulses being transmitted via a light guide or a light optic into the inner cavity, in particular the chamber, of the hollow needle, and emerge at a light exit side of the light guide or the light optics, such that vitreous material of the eye located in one chamber or the chamber formed inside of the hollow needle, which is fluidically communicating with a vitrectomy opening formed in the surgical device, is broken up by the exposure to the laser pulses, in particular cut; and
pressurizing the inner cavity, in particular the chamber, of the hollow needle with negative pressure, in particular in the range from 0.2 bar to 0.4 bar, preferably from 0.25 bar to 0.35 bar, more preferably from 0.26 bar to 0.33 bar, such that the glass body material broken up by the laser pulses is sucked away, and at the same time further vitreous material enters the chamber when the vitrectomy opening formed in the surgical device is positioned in the vitreous body of an eye.
